(19) United States
(12) Patent Application Publication
(10) Pub. No.: US 2002/0132846 A1
(43) Pub. Date: Sep. 19, 2002

(54) Use of gamma substituted gamma-butyrolactones to increase levels of their corresponding substituted gamma-hydroxybutyrate derivatives in humans

(76) Inventor: Caleb Stone, Venice, CA (US)

Correspondence Address: Caleb Stone, 2 Breeze Ave. # 108, Venice, CA, 90291, US

(21) Appl. No.: 10/081,516

(22) Filed: Feb. 21, 2002

Related U.S. Application Data
(60) Provisional application No. 60/271,616, filed on Feb. 26, 2001.

(51) Int. Cl. A61K 31/365

(52) U.S. Cl. 514/473

(57) ABSTRACT

This invention relates to a method of increasing C-4 substituted gamma-hydroxybutyrate levels in humans by administration of their respective gamma substituted gamma-butyrolactones, for the purposes of increasing human growth hormone levels, aiding sleep, and enhancing well-being.

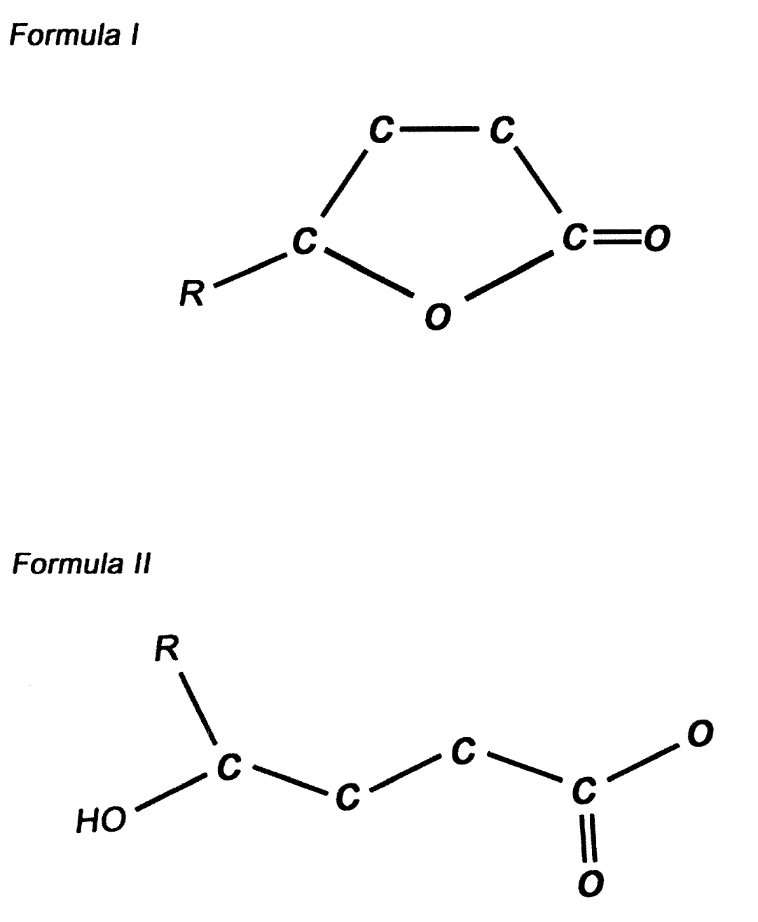

### FIELD OF THE INVENTION

This invention relates to a method of administering gamma substituted gamma-butyrolactones to increase levels of their corresponding substituted gamma-hydroxybutyrate derivatives in humans. These C-4 substituted gamma-hydroxybutyrate derivatives are closely related to the endogenous neuromodulator/neurotransmitter gamma-hydroxybute and one of them, the 4-methyl substituted compound, has been found to bind to the GHB receptor with greater affinity than gamma-hydroxybutyrate itself. Based upon structure activity relationships of gamma-hydroxybutyrate analogues, the others would be expected to bind strongly to the receptor as well. Thus, their exogenous administration would be expected to result in physiological and psychological effects similar to the administration of gamma-hydroxybutyrate. These effects include increases in human growth hormone levels, rapid and reliable onset of a sedation identical to human sleep—but with greater time spent in stage IV, and enhancement of well-being—including an increase in feeling of happiness, a decrease in feelings of depression, and reduction in anxiety.

### DESCRIPTION OF THE PRIOR ART

There are several pharmaceutical methods currently used to achieve the afore mentioned physiological and psychological effects. However, these all have significant disadvantages. Human growth hormone levels can be increased with recombinant human Growth Hormone injections, however these injections can be inconvenient and painful. The onset of sleep and the reduction of anxiety can be reliably achieved with benzodiazepines and their derivatives, however time spent in the most beneficial R.E.M. stage is reduced (1), and physical and psychological addiction can occur (2). Thus, benzodiazepines are not an ideal solution. Enhancement of well-being can be achieved with SSRI's, and novel antidepressants such as Wellbutrin and Effexor, however alleviation of symptoms often takes several weeks (3), thus these fall short as well.

With peroral administration, gamma-hydroxybutyrate has been shown to increase growth hormone levels by three fold (4), to reliably induce a sedation indistinguishable from normal human sleep (5), and to almost immediately enhance markers of well being—with no reports of physical or psychological addiction when used according to prescribed dosing levels and schedules.

### DESCRIPTION OF THE INVENTION

It was the object of this invention to discover naturally occurring, non-toxic, quickly metabolized precursors of C-4 substituted gamma-hydroxybutyrate derivatives with physiological and psychological effects similar to gamma-hydroxybutyrate. This is in order to be able to rapidly increase blood levels of said derivatives, therefore permitting peroral administration at a reasonable dose, and providing a rapid and reliable therapeutic response.

All of the proposed compounds are naturally occurring—found in things such beef, beer, cocoa, coffee, mushrooms, peaches, peanuts, wheat bread, heated butter, honey; and used as flavoring agents in candy, meat products, and baked goods—and are extremely non-toxic (6).

They are quite similar, structurally, to gamma-butyrolactone (possessing an alkyl chain in the gamma position rather than a hydrogen—see Drawing), a naturally occurring precursor to gamma-hydroxybutyrate in the brain, which rapidly forms the parent compound, upon oral ingestion, via lactonase catalyzed hydrolysis (7). In addition, The 4-methyl substituted gamma-hydroxybutyrate can be formed via hydrolysis from the 4-methyl substituted gamma-butyrolactone in the presence of a strong base, heat, and water (8). This is identical to a synthesis of gamma-hydroxybutyrate from gamma-butyrolactone. The lactone ring, where hydrolysis occurs, of all of the proposed structures are identical, thus they would be expected to rapidly be converted, upon oral ingestion, via lactonase catalyzed hydrolysis, into their respective C-4 substituted gamma-hydroxybutyrate derivatives The 4-methyl substituted gamma-hydroxybutyrate was shown to be 15% more potent at the receptor than gamma-hydroxybutyrate itself (8), Binding to the GHB receptor involves 2 binding sites. With gamma-hydroxybutyrate, the carboxylate end docks at one and the OH (alcohol) end docks at the other, with the distance between the two being a crucial factor in binding (9). The C-4 substituted gamma-hydroxybutyrates formed from our proposed gamma substituted gamma-butyrolactones are identical to gamma-hydroxybutyrate from carboxylate end to OH end, and structure activity studies have shown substitutions in the C-4 position to be well tolerated (9). Thus, all our proposed compounds would be expected to readily bind to the GHB receptor. Gamma-hydroxybutyrate exerts its effects through specific GHB receptors (10), thus oral administration of our gamma-substituted gamma-butyrolactones would be expected to result in conversion to their respective C-4 substituted gamma-hydroxybutyrates and, subsequently, to result in physiological and psychological effects quite similar to those produced by oral administration of gamma-hydroxybutyrate.

Oral gamma substituted gamma-butyrolactones can be given in daily doses of 0.1 ml to 5 ml. These daily doses can be divided into several subdoses, however a single nightly dose of 0.5 ml to 1.5 ml is most preferable. In addition to peroral administration, gamma substituted gamma-butyrolactones can be given via intravenous or peritoneal administration.

I claim:

1. A method of increasing C-4 substituted gamma-hydroxybutyrate levels in humans by administration of their respective gamma substituted gamma-butyrolactones, wherein the gamma substituted gamma-butyrolactone is represented by Formula I (see drawing), and the C-4 substituted gamma-hydroxybutyrate is represented by Formula II (see drawing)

2. The method of claim 1 wherein R is hydrogen, methyl, ethyl, propyl, butyl, pentyl, hexyl, heptyl, or octyl

3. The method of claim 1, for the purpose of increasing endogenous levels of human growth hormone.

4. The method of claim 1, for the purpose of aiding sleep.

5. The method of claim 1, for the purpose of enhancing well-being.

6. The method of claim 3, wherein “aiding sleep” refers to decreasing time to onset of sleep.

7. The method of claim 3, wherein “aiding sleep” means improving the quality of sleep.

8. The method of claim 3, wherein “enhancing well-being” means increasing feelings of happiness.

9. The method of claim 3, wherein “enhancing well-being” means reducing feelings of depression.

10. The method of claim 3, wherein “enhancing well-being” means reducing anxiety.

11. The method of increasing C-4 substituted gamma-hydroxybutyrate levels in humans according to claim 1, wherein the mode of administration is peroral.

12. The method of increasing C-4 substituted gamma-hydroxybutyrate levels in humans according to claim 1, wherein a peroral daily dosage of 0.1 ml to 5 ml is taken.
